(19) United States
(12) Patent Application Publication
(10) Pub. No.: US 2010/0035316 A1
(43) Pub. Date: Feb. 11, 2010

(54) METHOD OF BREWING ETHANOL

(76) Inventors: Wentao Li, Hong Kong (CN); Guipin Lv, Hong Kong (CN); Difei Zhao, Hong Kong (CN); Xiaolin Lei, Hong Kong (CN); Jianquan Li, Hong Kong (CN)

Correspondence Address: ROSENBERG, KLEIN & LEE, 3458 ELLICOTT CENTER DRIVE-SUITE 101, ELLICOTT CITY, MD, 21043, US

(21) Appl. No.: 12/310,227

(22) PCT Filed: Aug. 10, 2007
(86) PCT No.: PCT/CN2007/070455

§ 371 Date: Feb. 18, 2009

(30) Foreign Application Priority Data
Aug. 18, 2006 (CN) 200610111068.2

(51) Int. Cl. C12P 7/02

(52) U.S. Cl. 435/155

(57) ABSTRACT

A method of brewing alcohol, comprising at least the following steps: a grinding step of materials: grinding a mixture of the materials and water to obtain a mash containing material residues; a mixture-preparing step: mixing the mash containing material residues with yeast; a fermentation step: fermenting the mash containing material residues and the yeast to obtain a fermented mash containing material residues and alcohol; and a distillation step: distilling the fermented mash containing material residues and alcohol to obtain the alcohol. The mash containing material residues is used for mixing, then the mash containing material residues and the yeast is fermented, finally the mash containing the material residues and alcohol content is distilled. Not only is the sugar in the mash converted into alcohol, but also the other contents such as fiber and residual sugar in the material residues are converted into alcohol, which improves the yield of liquor by 20% to 25% and the production cycle is shortened.

### TECHNICAL FIELD

The present invention relates to a method of brewing alcohol, and in particular to a method of brewing alcohol by the fermentation of non-food crops.

### BACKGROUND OF THE INVENTION

It is known that since ancient times of China, there are methods of brewing alcohol by the way of fermenting the molasses. The molasses is obtained from the byproduct which is got after the beet sugar refining. The traditional method has a long history. However, in this traditional method of brewing alcohol, only the beet juice is used for brewing alcohol, which does not make the most use of the raw materials and further is longtime and complicated process.

In the modern methods of brewing wine, food crops are usually used without using the beet directly. Wine is obtained by the fermentation of food crops and starchy materials, and then distilled in the distillation tower to obtain alcohol for various uses.

As described above, the known methods have obvious defects which are desired to be overcome.

### SUMMARY OF THE INVENTION

Having outlined the state of the prior art and its attendant shortages, it is an object of the present invention to provide a method of brewing alcohol which uses non-food crops as materials directly, wherein the mash containing material residues is used for mixture-preparing, fermentation and then distillation, which improves the yield of liquor and makes the most use of the materials while generating a plurality of byproducts.

The above object of the present invention is achieved by the following technical solutions:

A method of brewing alcohol comprises at least the following steps:

a grinding step of materials: grinding a mixture of the materials and water to obtain a mash containing material residues;

a mixture-preparing step: mixing the mash containing material residues with yeast;

a fermentation step: fermenting the mash containing material residues and the yeast to obtain a fermented mash containing material residues and alcohol; and

a distillation step: distilling the fermented mash containing material residues and alcohol to obtain the alcohol.

Preferably, in the grinding step of materials, the materials are beets including dried beet and/or fresh beet.

Preferably, in the mixture-preparing step, the materials are mixed with the yeast at a ratio of 1:200-400 by weight.

Preferably, in the mixture-preparing step, the pH value, concentration and temperature of the mash containing material residues are regulated before adding the yeast.

Preferably, in the mixture-preparing step, the pH value of the mash containing material residues is regulated into 5 to 6.

Preferably, in the mixture-preparing step, the concentration is regulated into a ratio of the materials to the water being 1:0.8-5 by weight.

Preferably, in the mixture-preparing step, the temperature of the mash containing material residues is regulated into 32° C. to 35° C.

Preferably, in the fermentation step, the mash containing material residues and the yeast is fermented continuously or intermittently.

Preferably, the distillation step comprises at least the following steps: a coarse distillation step: distilling the fermented mash containing material residues and alcohol coarsely; and a fine distillation step, distilling the mash after the coarse distillation step to obtain the alcohol.

With the above-described technical solutions, the present invention is of the following advantages:

1. In the alcohol production process, the mash containing material residues is used for mixture-preparing, fermentation and then distillation, thereby not only is the sugar in the mash converted into alcohol, but also the other contents such as fiber and residual sugar in the material residues are converted into alcohol, which improves the yield of liquor by 20% to 25% and makes the most use of the materials;

2. The use of non-food crops such as beet for the alcohol production caters for the development of this field to solve the problems including non-biomass energy source, three-dimensional rural issues concerning agriculture, countryside and farmers, and the conflict between the alcohol production and the eating if food crops are used;

3. The saccharifying, liquescence and fermentation processes are performed in one step and one container, which in traditional method different containers are used in these three processes;

4. The materials may be dried or fresh, that is, the production may be performed in an universal device for the fresh and dried materials;

5. Safe residues are generated during the production, which realizes the recycling model to achieve the aim of resource [symbol] product [symbol] recycling resource, wherein:

(1) the liquid separated from the coarse distillation is filtered to obtain a filtrate which is then sent to the mixture-preparing step for regulating the concentration, while the others may be used for producing methane and provide the heat energy with other energy such as coal and gas for the production process;

(2) all of the liquids including the liquid of lees liquid discharged from coarse distillation, condensing water after distillation, and alkalescent water after washing containers and pipes may be recycled after simple treatment, for use in the mixture-preparing step, boiler, environment, washing, etc.;

(3) solid residues obtained from the coarse distillation may be used as feedstuff, or degradable materials, etc. and for producing high-grade fertilizer together with fermented sludge;

(4) the carbon dioxide generated during the fermentation may be recycled to be as industrial product.

6. The fermentation process is completely performed under the yeast without occurring high temperature, thus no device for reducing temperature by spraying water is necessary for the fermentation tank and it is good for the generation of safe residue;

7. the production cycle is shortened to 20-30 hours, while investment is reduced with high production efficiency.

### BRIEF DESCRIPTION OF THE DRAWINGS

FIG. 1 is a flowchart of the method of brewing alcohol according to an embodiment of the present invention.

### DETAILED DESCRIPTION OF THE INVENTION

The present invention will further described in the following embodiments accompanying with the drawings.

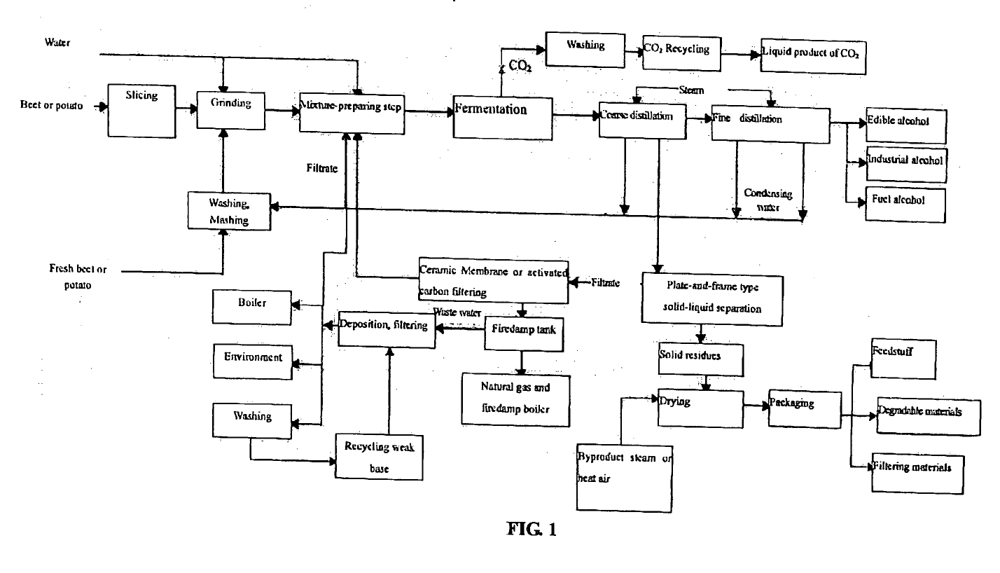

Referring to FIG. 1, an example using beets as materials is shown.

The beet is mixed with water to proceed with grinding to obtain mash containing beet residues. The beet may be dried beet after slicing and drying, or fresh beet after washing and smashing, or the mixture of dried and fresh beets.

In the mash containing material residues, the pH value, concentration and temperature of the mash containing beet residues are regulated. The pH value of the mash containing beet residues is regulated into 5 to 6 by adding water and additives. The concentration is regulated into a ratio of the beets to the water being 1:0.8-5 by weight. The temperature of the mash containing beet residues is regulated into 32° C. to 35° C. Then, different yeast is added at different ratio according to different materials. The beets are mixed with yeast at a ratio of 1:200-400 by weight, and thus the mixture-preparing step is finished.

The mash containing beet residues is then fermented intermittently, during which the mash is blended evenly. All fermentation stages of the fermenting mash are performed in one fermentation tank during the intermittent fermentation. Alternatively, continuous fermentation may be adopted, and in the continuous fermentation the mash containing beet residues is also used and the mash is also blended evenly.

Then, the fermented mash is distilled to obtain alcohol including edible alcohol, industrial alcohol and fuel alcohol. The distillation includes coarse distillation and fine distillation. Solid-liquid separation may be performed before the coarse distillation, however in this embodiment the mash without removing the beet residues is distilled coarsely in the coarse distillation tower to improve the alcohol content, while in the fine distillation, steam is used for the fine distillation of the mash after coarse distillation, and the byproduct steam or heat air in the production processes may be utilized.

In the steps of the production processes, a large amount of useful safe residues is generated and may be recycled for use in the processes or other use.

In the fermentation step, the CO₂ may be recycled by washing many times to obtain liquid products of CO₂.

In the coarse distillation step, the lees liquid obtained from the coarse distillation is proceed with solid-liquid separation, where the plate-and-frame filter press, the centrifuge or other apparatus may be used in solid-liquid separation. The separated liquid may be sent to the mixture-preparing step to adjust the concentration, and the liquid after filtering may be sent into firedamp tank for fermentation to produce firedamp which may be then used for generating heat energy for use in the production processes. The waste water in the firedamp tank after treatments including deposition by weak base, to recycle the clean water, and the weak base may be also recycled. The solid residues separated from the lees liquid may be used as feedstuff, degradable materials, or filtering materials, after drying and packaging.

The condensing water after distillation and the alkalescent water after washing the containers and pipes may be recycled by simple treatment to be used for washing materials, boilers, or other use.

Preferably, the materials used in the present invention are beets. Other materials such as fresh or dried sweet potato or white potato may also be used.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A method of brewing alcohol, comprising at least the following steps:
a grinding step of materials: grinding a mixture of the materials and water to obtain a mash containing material residues;
a mixture-preparing step: mixing the mash containing material residues with yeast;
a fermentation step: fermenting the mash containing material residues and the yeast to obtain a fermented mash containing material residues and alcohol; and
a distillation step: distilling the fermented mash containing material residues and alcohol to obtain the alcohol.

2. The method of claim 1, wherein in the grinding step of materials, the materials are beets including dried beet and/or fresh beet.

3. The method of claim 1, wherein in the mixture-preparing step, the materials are mixed with the yeast at a ratio of 1:200-400 by weight.

4. The method of claim 1, wherein in the mixture-preparing step, the pH value, concentration and temperature of the mash containing material residues are regulated before adding the yeast.

5. The method of claim 4, wherein in the mixture-preparing step, the pH value of the mash containing material residues is regulated into 5 to 6.

6. The method of claim 4, wherein in the mixture-preparing step, the concentration is regulated into a ratio of the materials to the water being 1:0.8-5 by weight.

7. The method of claim 4, wherein in the mixture-preparing step, the temperature of the mash containing material residues is regulated into 32° C. to 35° C.

8. The method of claim 1, wherein in the fermentation step, the mash containing material residues and the yeast is fermented continuously or intermittently.

9. The method of claim 1, wherein the distillation step comprises at least the following steps: a coarse distillation step: distilling the fermented mash containing material residues and alcohol coarsely; and a fine distillation step: distilling the mash after the coarse distillation step to obtain the alcohol.
